(19) United States
(12) Patent Application Publication
(10) Pub. No.: US 2024/0001005 A1
(43) Pub. Date: Jan. 4, 2024

(54) BLOOD PRODUCT FOR PREVENTING SURGICAL ADHESION

(71) Applicant: REAPPLIX A/S, Birkerød (DK)

(72) Inventors: Rasmus LUNDQUIST, Brønshøj (DK); Niels Erik HOLM, Birkerød (DK); Morten Rosenberg ESKILDSEN, Allerød (DK)

(73) Assignee: REAPPLIX A/S, Birkerød (DK)

(21) Appl. No.: 18/250,049

(22) PCT Filed: Oct. 21, 2021
(86) PCT No.: PCT/EP2021/079277

§ 371 Date: Apr. 21, 2023

Related U.S. Application Data
(60) Provisional application No. 63/094,694, filed on Oct. 21, 2020.
(60) Provisional application No. 63/094,695, filed on Oct. 21, 2020.
(60) Provisional application No. 63/094,700, filed on Oct. 21, 2020.

(51) Int. Cl. A61L 31/04; A61L 31/16

(52) U.S. Cl. CPC A61L 31/046; A61L 31/16; A61L 2300/64

(57) ABSTRACT

A method for preventing surgical adhesion between surfaces that are to be prevented uses a blood product having fibrin. The blood product can be a singled layered blood product having components from plasma, components added to initiate a coagulation process, and components created during the coagulation process. The products and methods can be used for preventing surgical adhesion, for stopping or reducing bleeding in an open surgical wound, and for treating surgical wounds.

### CROSS-REFERENCE TO RELATED APPLICATIONS

This application is a National Stage Application filed under 35 U.S.C. § 371 of PCT Application No. PCT/EP2021/079277, filed Oct. 21, 2021, which claims priority to and the benefit of U.S. Provisional Patent Application. No. 63/094,694, filed Oct. 21, 2020, U.S. Provisional Patent Application. No. 63/094,695, filed Oct. 21, 2020, and U.S. Provisional Patent Application. No. 63/094,700, filed Oct. 21, 2020, the entire contents of all of which are incorporated by reference herein.

### BACKGROUND OF THE DISCLOSURE

### 1. Field of the Disclosure

The present disclosure relates to surgery, in particular to products and methods for preventing surgical adhesion, for stopping or reducing bleeding in an open surgical wound, and for treating surgical wounds.

### 2. Description of Related Art

The human coagulation system can stop bleeding and initiate healing. The function of the system to stop bleeding is well known and extensively investigated. However, the importance of coagulation products in the initiation of healing has been less investigated.

Blood products, such as fibrin sealants and platelets concentrate, are produced by isolating the platelet rich plasma (PRP) from anti-coagulated whole blood. The presence of platelets and plasma partly imitates the natural human coagulation system upon thrombin activation. This leads to a platelet containing autologous concentrate of growth promoting factors in a fibrin matrix. Such a composition can be used for covering wound surfaces and is claimed to initiate healing.

“Platelet-rich fibrin (PRF): A second-generation platelet concentrate. Part I: Technological concepts and evolution, Oral Surg Oral Med Oral Pathol Oral Radiol Endod 2006; 101:E37-44” by David M. Dohan et al describes how to prepare a platelet rich solid fibrin network from whole blood without adding any additives or reagents. The PRF protocol is: A blood sample is taken without anticoagulant in 10-mL glass tubes or glass coated plastic which are immediately centrifuged at approximately 400 g for 10 minutes. The absence of anticoagulant implies the activation in a few minutes of most platelets of the blood sample in contact with the glass tube walls and initiation of the coagulation cascades. Fibrinogen is initially concentrated in the plasma part of the tube before the circulating thrombin transforms it into fibrin. A fibrin clot is then obtained in the middle of the tube, extending from the upper part of the red corpuscles at the bottom of the tube to the acellular plasma at the top. Platelets are trapped massively in the fibrin meshes. The success of this technique entirely depends on the speed of blood collection and transfer to the centrifuge. Indeed, without anticoagulant, the blood samples start to coagulate almost immediately upon contact with the tube glass, and it takes a minimum of a few minutes of centrifugation to concentrate fibrinogen in the middle and upper part of the tube. Quick handling is the only way to obtain a clinically usable PRF clot. If the duration required to collect blood and launch centrifugation is overly long, failure will occur: The fibrin will polymerize before red cells has been separated and result in entire tube to be one blood clot. In conclusion, the PRF protocol makes it possible to collect a fibrin clot charged with serum and platelets. By removing the clot from the tube, manually cutting of the red cells part, and manually driving out the fluids trapped in the fibrin matrix (serum), practitioners will obtain autologous fibrin membranes.

However, this fibrin network includes some red thrombus containing a substantial part of red blood cells, which have to be manually cut off. In this step, part of the PRF will also be cut off. Furthermore, the components of the produced fibrin network, such as fibrin, leukocytes and thrombocytes, are arbitrary distributed and enmeshed within the product. The recovery of leukocytes is not described and at the low g force used, the recovery of leukocytes is low and as result, some will be located in the red cell part and will be lost. The enmeshment of cells within the fibrin leads to absent or slow release of these cells and thereby inhibits the contact-dependent anti-microbicidal potential of the included leucocytes.

“Oral Surg Oral Med Oral Pathol Oral Radiol Endod 2006; 101:E37-44 and 2006; 101:E45-50” by Dohan et al describes a network that does not represent a platelet concentrate in a shape and structure, which is directly applicable for covering wound surfaces. To obtain a shape and form/rigidity useable for covering wound surfaces and prevent red blood cell inclusion, the known platelet rich solid fibrin network will have to be reshaped manually and compressed. Furthermore, the method comprises several steps and cannot be prepared in one closed system and is therefore not convenient for clinical use.

“Cell separation in the buffy coat. Biorheology. 1988; 25(4):663-73” by Sutton et al describes how anti-coagulated full blood will separate into several layers upon centrifugation or passive sedimentation; Red blood cells, leukocytes and platelets (=buffycoat) and plasma. Further, by using centrifugation force of 10000 g for 10 minutes and using a float of density of 1.053, the buffy coat can be fixed by Glutaraldehyde, and removed for investigation; however, this cannot be used clinically due to the toxicity of the Glutaraldehyde. Several methods for extracting the buffycoat from anti-coagulated blood exists including the use of density defined substances (i.e. Lymphoprep). In addition to the need for anti-coagulated blood the extracted cells will be suspended—and mixed (disorganized)—in the plasma that inevitably will be included.

EP 1637145A describes a method of filtration of cells from a suspension (e.g. blood cells including platelets and leukocytes) through a sheet like porous membrane, leaving the cells in the membrane as described. The sheet porous material can be prepared from fibrin. However, no layered structure is obtained, the cells are trapped in depth in the porous material and the use of allogeneic fibrin raises the risk of cross infection from other humans. Furthermore, the method comprises several steps and cannot be prepared in one closed system and is therefore not convenient for clinical use.

WO2010/020254 discloses a multilayered blood product comprising components from whole blood, especially fibrin, thrombocytes and leukocytes. The multilayered blood product is used for treatment of a wound where the blood product can improve healing of the wound.

Adhesions can form as a natural part of the body's healing process after surgery. The term “adhesion” is applied when the tissue is generated and extends from within one tissue across to another, usually across a virtual space such as the peritoneal cavity. Adhesion formation post-surgery typically occurs when two injured surfaces are close to one another. This often causes inflammation and causes fibrin deposits onto the damaged tissues. The fibrin then connects the two adjacent structures where damage of the tissues occurred. The fibrin acts like a glue to seal the injury and builds the fledgling adhesion, said at this point to be “fibrinous.”

While some adhesions do not cause problems, others can prevent muscle and other tissues and organs from moving freely, sometimes causing organs to become twisted or pulled from their normal positions.

There is therefore a desire to prevent or reduce adhesions to form after surgery.

Also, there is a need for improved surgical methods and products enabling such methods.

### SUMMARY OF THE DISCLOSURE

The object of the disclosure is to provide blood products for use in surgical methods for e.g. reducing adhesion, for stopping and reducing bleeding from open surgical wounds, and for treating surgical wounds in order to improve healing and reduce generation of scars.

It is also an object of the disclosure to provide a new method for reducing surgical adhesion.

In a first aspect the present disclosure provides a method for preventing or reducing adhesion during surgery between surfaces where adhesion is to be prevented, comprising the steps of
a. identifying surfaces that during surgery will be located close to each other, which surfaces and not intended to adhere to each other; and
b. applying a blood product comprising a high concentration of fibrin at one or all of the identified surfaces.

Surfaces where adhesion is to be prevented is according to the disclosure understood as surfaces that are exposed during surgery, and which will after the surgery be located so close to each other that there is a risk for that adhesion between the surfaces will be formed during healing after the surgical incision; which surfaces are not in the natural state attached to each other and therefore is adhesion of the surfaces not desired during healing and afterwards. The surfaces in question are in particular surfaces that are injured during the lesion that is the reason for the surgery or injured during the surgery procedure itself, because the healing process will start from these surfaces.

It is within the skills of the average surgeon to identify such surfaces.

When the surfaces where adhesion is to be prevented have been identified blood product comprising a high concentration of fibrin is applied to the surfaces. The blood product can be applied as a single sheet of the blood product covering the whole identified surface or it can be applied as a number of independent sheets that are applied adjacent to each other or with a slight overlap.

In one embodiment the blood products are prepared by centrifugation processes, and in this embodiment, the blood product is obtained as round sheets, determined by the size and form of the cross-section of the centrifuge tubes used for the centrifugation process. Consequently, in this embodiment the identified surfaces are covered by one or more round sheets of the blood product until the complete surface is covered.

Adhesion takes place between two or more surfaces that not in the natural state are attached to each other and adhesion can according to the disclosure be reduced or prevented by applying the blood product or one, two or all the identified surfaces.

After the blood product has been applied on the identified surfaces and the surgery is over the surgical wound is closed, as known in the art, and healing process begins. According to the method of the disclosure, the healing will proceed will no adherence between the surfaces where the blood product has been application, or at least reduced adherence compared with a similar surgical wound where the blood product has not been applied according to the disclosure.

The blood product is generated from a blood sample from a suitable donor, preferable from the patient itself.

In a preferred embodiment, the blood product comprises fibrin that have resistance against fibrinolysis, which secures that the blood product will remain intact in place and exerts its function longer compared with a similar blood product where the fibrin do not have resistance against fibrinolysis. One method for obtaining a blood product comprising fibrin that have resistance against fibrinolysis is to allow fibrin to polymerize in 3 dimensions and compress the product to a thinner layer, e.g. by centrifugation.

In one preferred embodiment, the blood product is reinforced by another structure e.g. an inert net such as a synthetic net made primarily of polyglycolic acid, for example Vicryl© net.

### The Blood Product

In a second aspect the present disclosure provides a blood product for use in a method for preventing or reducing adhesion during surgery between surfaces where adhesion is to be prevented, comprising the steps of
a. identifying surfaces that during surgery will be located close to each other, which surfaces are not intended to adhere to each other; and
b. applying a blood product comprising a high concentration of fibrin at one or all of the identified surfaces.

In a third aspect the present disclosure provides a blood product for use in a method for surgical wound treatment comprising the steps of
c. Cleaning the wound from foreign material;
d. Removing fluids such as blood;
e. Applying a blood product to the wound, so the blood product covers the complete wound bed ensuring contact between the blood product and the intact skin boundary surrounding the wound; and
f. Covering the wound with a primary sheet;
wherein the blood product is selected among:
i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the wound; and
ii. single layered blood products consisting of components from plasma, components added to initiate a coagulation process and components created during the coagulation process.

In a fourth aspect the present disclosure provides blood product for use in a method for stopping or reducing bleeding in an open surgical wound comprising the steps of
g. identifying the bleeding source;
h. removing excess fluids such as blood from the area around the bleeding source;
i. applying a blood product to the bleeding source; and
j. maintaining the blood product in close contact with the bleeding source until bleeding is ceased or reduced;
wherein the blood product is selected among:
i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the bleeding source; and
ii. single layered blood products consisting of components from plasma, components added to initiate a coagulation process and components created during the coagulation process.

The blood product comprises fibrin from whole blood. Further, it can also comprise blood-cells like white cells and platelets. Further, the distribution of white cells and platelets in the blood product differs.

The blood product has preferably a maximum width/thickness-ratio of 1, 2, 3, 4, 5, or up to 10, 15, 20, 25, 30, 40, 50, 60, 70, 80, 90, 100 or even up to 500, where the width is measured along the layers of the blood product and the thickness is measured perpendicular to the blood product. In an embodiment the width/thickness ratio is in the range from 10-500, from 15-200 or from 20-200.

The blood product is self-supporting, compact, and solid, and the blood product has a structure, such that the blood product is directly applicable for the intended use.

In one embodiment the blood product comprises mainly fibrin isolated from whole blood.

In another embodiment the blood product comprises mainly fibrin and platelets isolated from whole blood. The blood product is self-supporting, compact, and solid, and the blood product has a structure, such that the blood product is directly applicable for the intended use.

In another embodiment the blood product comprises mainly fibrin and platelets isolated from whole blood were the concentration of fibrin and platelets differs in the depth/thickness of the product such that the fibrin concentration is high on one side of the product and low on the other side. Likewise, the platelets concentration is high on the opposite side of the high fibrin concentration and low on the high fibrin concentration side.

In yet another embodiment the blood product comprises mainly fibrin, platelets and white cells isolated from whole blood were the concentration of fibrin, platelets and white cells differs in the depth/thickness of the product such that the fibrin concentration is high on one side of the product and low on the other side. Likewise, the platelets and white cells concentration is high on the opposite side of the high fibrin concentration and low on the high fibrin concentration side.

In another embodiment the blood product solely consists of components from plasma, added calcium chloride and components created during the coagulation process. Hereby, a single layered blood product is provided that solely consists of components from plasma, meaning that no additives except for calcium chloride are added to the product, and that the blood product is directly derivable from the plasma fractions of whole blood.

In another embodiment the blood product includes reagents, that may prevent/delay fibrinolysis.

In another embodiment the blood product includes reagents that may stimulate the coagulation during manufacturing, such as Kaolin.

In another embodiment the blood product includes other material to increase the physical strength of the product, such as a Vicryl net.

The blood product according to the disclosure can be prepared in advance of the surgical procedure and stored until use. For longer term storage it is preferred to dry the blood product immediately after preparation. The dry blood products can thereafter by unpacked and used during a subsequent surgical procedure.

Premade and stored blood products, e.g. dried blood products, has the clear advantage that an adequate supply can be prepared and put on stock before the surgical procedure in order to avoid the need for staff and equipment for preparation of the blood product during the surgical procedure.

Under all circumstances it is important to maintain sterility during the preparation and storage of any blood product in order to avoid contamination and undesired growth during storage. Alternatively, the premade blood products can be sterilized using methods that do not denature fibrin, or only denature fibrin in a smaller extent, such as gamma radiation or ethylene oxide treatment.

In a preferred embodiment, the blood product is the multilayered blood product disclosed in WO 2010/020254 A1, which is incorporated by reference, i.e., the blood product comprises components from whole blood, especially fibrin, thrombocytes and leukocytes, the blood product comprising a first layer, a second layer and a third layer, the second layer being adjacent to the first layer and the third layer, the first layer defining a first outer surface of the blood product and the third layer defining a second outer surface of the blood product, the first layer comprising a majority of leukocytes, the second layer comprising a majority of thrombocytes and the third layer comprising a majority of fibrin. Hereby, a blood product with a multilayered structure is provided, each layer provides different functionality due to each of the layer's different composition. The blood product is self-supporting, compact and solid, and the blood product has a structure, such that the blood product is directly applicable for the intended use. By majority is meant, that a component, such as fibrin, thrombocytes or leukocytes, comprises at least 50%, 55%, 60%, 65%, 70%, 75%, 80%, 85%, 90%, 95%, 98%, 99% or even 100%, or any interval that can be defined from combinations of these mentioned percentages, volume- and/or mass wise of the respective layer and/or volume- and/or mass wise of the blood product. The first, the second and the third layer are each continuous and/or substantially parallel to each other, e.g. forming a stratified and/or multilayered blood product. The blood product preferably consists of three layers, e.g. a first layer comprising a majority of leukocytes, a second layer comprising a majority of thrombocytes and a third layer comprising a majority of fibrin. The blood product has preferably a maximum width/thickness-ratio of 1, 2, 3, 4, 5, or up to 10, 15, 20, 25, 30, 40, 50, 60, 70, 80, 90, 100 or even up to 500, where the width is measured along the layers of the blood product and the thickness is measured perpendicular to the layers of the blood product. In an embodiment the width/thickness ratio is in the range from 10-500, from 15-200 or from 20-200.

In one embodiment, a majority of the leukocytes comprised in the blood product are comprised in the first layer. Hereby, a blood product is provided where the majority of the leukocytes comprised in the entire blood product is comprised in the first layer.

In another embodiment, a majority of the thrombocytes comprised in the blood product are comprised in the second layer. Hereby, a blood product is provided where the majority of the thrombocytes comprised in the entire blood product are comprised in the second layer.

In another embodiment, a majority of the fibrin comprised in the blood product is comprised in the third layer. Hereby, a blood product is provided where the majority of the fibrin comprised in the entire blood product is comprised in the third layer.

In other embodiments, the blood product solely consists of components from whole blood. Hereby, a blood product is provided that solely consists of components from whole blood, meaning that no additives are added to the whole blood and/or the blood product, and that the blood product is directly derivable from whole blood, fractions of whole blood or combinations of fractions of whole blood.

Preferably, the blood product is autologous.

The blood product can be flexible. Hereby, a blood product is provided that can withstand applied stress during normal use without rupturing. Due to the flexibility of the blood product, the blood product conforms most continuous contours whereto the blood product is applied.

In another embodiment, the blood product further comprises a first substance chosen from a group comprising fibroblasts, keratinocyte cells and hyaluronic acid. Hereby, a blood product is provided which includes additional cells known to be important for skin regeneration and thereby further improves the healing potential of chronic wounds, especially wounds in areas with low or unviable adjacent tissue. Hyaluronic acid, a known component of skin, has the potential to increase the water binding capacity of the blood product as well as increase the potential for incorporation/infiltration of the blood product in areas of tissue loss.

The disclosure also relates to, a blood product for therapeutic use and/or a therapeutic method for a blood product.

The disclosure also relates to a method of manufacturing a blood product with a medicament for therapeutic use.

The disclosure also relates to a blood product for treatment of a wound and/or a method for treatment of a wound with a blood product in order to reduce healing time and improve visual impression.

The disclosure also relates to a blood product for stopping or reducing bleeding in an open surgical wound.

The disclosure also relates to for a method of manufacturing of a medicament having a blood product for treatment of a wound to reduce healing time and improve visual impression. Hereby, a blood product is provided which is particularly suitable for manufacturing of a medicament for treatment of a surgical wound.

By applying the first outer surface defined by the first layer against the bleeding source, bleeding can be reduced or stopped, as the first layer comprises a majority of leukocytes, which are the first active cells and the second layer comprises a majority of thrombocytes which promotes coagulation.

The third layer of the blood product comprises a majority of fibrin, and thus the second outer surface of the blood product provides an effective barrier of the bleeding site and can form a physical barrier that can reduce bleeding; the third layer can furthermore comprise growth promoting factors, which is released over time.

Preparation of the Blood Product

The blood product can be prepared from a volume of whole blood, the method comprising the following steps: a) placing the volume of whole blood in a container means, the container means comprising a first material defining an inner surface in which the whole blood is in contact with, b) separating the whole blood into erythrocytes, serum and blood product by a centrifugal force acting on the whole blood placed in the container means, whereby the whole blood separates into layers comprising erythrocytes, blood product and serum due to the differences in densities between the erythrocytes, blood product and serum, the blood product comprising leukocytes and thrombocytes, the applied centrifugal force being at least 1000 times greater than the gravity force, e.g. g, acting on the whole blood, and d) removing the blood product from the container means.

In other embodiments the blood product is prepared from the plasma fraction of blood by a method comprising following steps: a) placing a volume of plasma in a container means; b) adding a component that initiate a coagulation process and letting the coagulation process proceed; c) applying a centrifugal force being at least 1000 times greater than the gravity force in a sufficient time to compact the coagulated material as a single layered product; and d) removing the blood product from the container means. Alternatively, step b) is performed before step a).

Components that initiate a coagulation process are in general hydrophilic and/or polar surfaces such as kaolin, glass, iron, etc. Further, it is important that Calcium ions are available for the process, so a typical way of initiating coagulation is adding calcium chloride and exposing the plasma to a hydrophilic and/or polar surface, e.g. an object composed of such a material or even the wall of the container means.

In a preferred embodiment the blood product is prepared according to the methods disclosed in WO 2010/020254, as outlined below.

The blood product can be prepared from a volume of whole blood, the method comprising the following steps: a) placing the volume of whole blood in a container means, the container means comprising a first material defining an inner surface in which the whole blood is in contact with, b) activating coagulation of the whole blood, c) separating the whole blood into erythrocytes, serum and blood product by a centrifugal force acting on the whole blood placed in the container means, whereby the whole blood separates into layers comprising erythrocytes, blood product and serum due to the differences in densities between the erythrocytes, blood product and serum, the blood product comprising fibrin, leukocytes and thrombocytes, the applied centrifugal force being at least 1000 times greater than the gravity force, e.g., acting on the whole blood, and d) removing the blood product from the container means. Hereby, a method is provided whereby a blood product is derivable from whole blood. The method can be processed in one cycle in a closed system, since there is no need for an isolation of the erythrocytes; however, the method can also be performed using an isolation of the erythrocytes. The yield of the method for extracting fibrin from the whole blood is at least above 10%, 20%, 30%, 40%, 50%, 60%, 70%, 80%, 90%, 95%, 97%, 98%, 99% or even 100%, while the yield of the method for extracting leukocytes from the whole blood is at least above 10%, 20%, 30%, 40%, 50%, 60%, 70%, 80%, 90%, 95%, 97%, 98%, 99% or even 100%, while the yield of the method for extracting thrombocytes from the whole blood is at least above 10%, 20%, 30%, 40%, 50%, 60%, 70%, 80%, 90%, 95%, 97%, 98%, 99% or even 100%. The obtainable blood product has a volume less than 30%, 20%, 15%, 10% or even less than 5% of the volume of the whole blood. Hereby, a method is provided whereby a blood product is obtainable by e.g. centrifugation giving rise to a centrifugal force. The applied centrifugal force is at least 1000, 2000, 3000, 4000, 5000, 6000, 7000, 8000, 9000, 10000, 11000, 12000, 13000, 14000, 15000, 16000, 17000, 18000, 19000 or even times greater than the gravity force, e.g., acting on the whole blood, or the applied centrifugal force is within any interval that can be defined from combinations of the mentioned numbers. The time the centrifugal force is applied for at least 30 seconds, 40 seconds, 50 seconds, 60 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes, or the centrifugal force is applied within any interval that can be defined from combinations of the mentioned numbers.

The yield of the method for extracting fibrin from the whole blood is preferably at least 60%, or more.

The yield of the method for extracting leukocytes from the whole blood is preferably at least 50%, or more.

The yield of the method for extracting thrombocytes from the whole blood is preferably at least 60%, or more.

In one aspect of the method, the centrifugal force is applied for at least 30 seconds. The time the centrifugal force is applied for at least 30 seconds, 40 seconds, 50 seconds, 60 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes or 30 minutes, or the centrifugal force is applied within any interval that can be defined from combinations of the mentioned numbers.

In one embodiment, the blood product solely consists of components from whole blood. Hereby, a method is provided whereby a blood product is derivable from whole blood, thus solely consisting of components from whole blood. Thus, the method is performed without adding any additives to the whole blood and/or the blood product.

In another aspect of the method, the coagulation in step b) is activated by the first material defining the inner surface. Hereby, a method is provided in which the coagulation is initiated when the whole blood is brought in contact with the inner surface, thus it can be avoided to add any object or the like to the whole blood to initiate coagulation.

In another aspect of the method, the coagulation in step b) is activated by exposing the whole blood to an object, such as a glass bead. Hereby, a method is provided in which the coagulation is initiated when the whole blood is brought in contact with the object added to the whole blood. Hereby, the coagulation can be initiated at a chosen point of time, which is optimal for the method.

In another aspect of the method, the first material of the inner surface of the container means is chosen from a group comprising of polypropylene, polyethylene, polycarbonate, polyamide, acrylonitrile butadiene styrene, styrene, modified styrene, polyurethane and other polymer materials. The polymers in the mentioned group can furthermore be glass-filled. Hereby, a method is provided where a container means with an inner surface of a first material can be typical test tubes or the like made from all kinds of polymers, metal or glass. The material can also be chosen so the material property provides a minimal adhesive force/friction between the blood product and the inner surface. Polyamide and polyurethane are preferred as these materials initiates coagulation within a preferred level of activation which is higher than the level obtained using other polymers.

In another aspect of the method, the inner surface of the container means is surface treated, e.g. coated, in order to lower friction between the blood product and the inner surface of the first material. Hereby, a method is provided where a container means with an inner surface of a first material can be typical test tubes or the like made from all kinds of polymers, metal or glass. The inner surface can be surface treated and/or coated to obtain a minimal adhesive force/friction between the blood product and the inner surface.

In another aspect of the method, the centrifugal force is greater than an adhesive force acting between the inner surface and the blood product. Hereby, a method is provided where the centrifugal force is dominant as compared to the adhesive force, which secures a well-defined layered structure of the blood product. The centrifugal force can be at least 10, 100, 1000, 5000, 10000, 20000, 50000, 100000, 1000000 or even 10000000 times greater than the adhesive force, or the centrifugal force is within any interval that can be defined from combinations of the mentioned numbers. In another aspect of the disclosure, the centrifugal force and centrifugation time is of such strength that the adhesive force acting between the inner surface and the fibrin is broken/released and thereby allowing the fibrin layer to be compacted/compressed. Hereby, a method is provided where the centrifugal force is dominant as compared to the adhesive force, which secures a well-defined layered structure of the blood product. The centrifugal force needed to release the adhesion to the wall will depend on the fibrin density. The fibrin density will depend on several factors including coagulation activation, fibrin concentration, time, etc. The centrifugation force can be at least 10, 100, 1000, 5000, 10000, 20000, 50000, or even 100000, g, or the centrifugal force is within any interval that can be defined from combinations of the mentioned numbers.

In another aspect of the method, the blood product adhering to the inner surface is detached from the inner surface, at least once, during step c). Hereby, a method is provided where the possible adhesion of the blood product to the inner surface can be dealt with by separating the blood product at least once during step c). The separation can be done by mechanical means such as by cutting or the like. The compacting of the fibrin can then be performed at lower g-force as the g-force does not need to release the fibrin from the wall.

In another aspect, the method further comprises a compacting step, where the blood product is compacted by a compacting means, such as a filter placed in the container means. Hereby, a method is provided where the blood product can be compacted by e.g. a filter. The filter can be placed fixed or movable in the container means and the filter can be used to isolate the blood product.

In another aspect, the method further comprises an isolation step, where the erythrocytes are isolated from the blood product during step c). Hereby, a method is provided where the erythrocytes are isolated from the serum and the blood product during the method.

In another aspect, the method further comprises a washing step where the blood product is washed, so substantially all erythrocytes and/or serum attached to the blood product are detached. Hereby, a method is provided so the blood product is substantially clean from other components, that of the blood product itself. The serum formed during the centrifugation can be used as a washing fluid.

In another aspect of the method, the container means is a tube comprising an open end, closable by a detachable lid, and a closed end. Hereby, a method is provided where a standard test tube or the like can be used to perform the method.

In another aspect of the method, step b) precedes step a). Hereby, the coagulation can be activated before the whole blood is placed in the container means, thus the container means does not need to comprise any coagulation activator and/or the whole blood does not need to comprise a coagulation activator when the whole blood is placed in the container means. The coagulation can as an example, be activated during blood drawing by placing glass beads in the blood drawing tubing or choosing a tubing material that will activate blood. Thus, it can be avoided to add any object or the like to the whole blood to initiate coagulation.

In another aspect of the method, step b) occurs at least 1 minutes before step a). Hereby a method is provided where very fast handling is not necessary. Step b) can occur at least 30 seconds, 40 seconds, 50 seconds, 60 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes or even 20 minutes before step a). The level of activation of coagulation in step b) can also be controlled by the method. This allows the time between step b) and a) to be prolonged as the cell separation in step c) has to occur before fibrin levels is sufficient to inhibit cell separation.

In another aspect of the method, step b) occurs concurrently with step c). Hereby, the coagulation can be activated at an optimum point of time for the method during step c). The coagulation can be activated/initiated by a coagulation activator integrated into the container means, and/or by using a container material that will activate the blood. The coagulator material can be an object, such as glass beads, added to the whole blood in the container means.

In another aspect of the method, a first substance chosen from a group comprising fibroblasts, keratinocyte cells and hyaluronic acid is added to the whole blood. Hereby, a blood product is obtainable which includes additional cells known to be important for skin regeneration and thereby further improves the healing of chronic wounds, especially wounds in areas with low or unviable adjacent tissue. Hyaluronic acid, a known component of skin, has the potential to increase the water binding capacity of the blood product as well as increase the potential for incorporation/infiltration of the blood product in areas of tissue loss.

In another aspect of the method, the compacting means, such as a filter, has a first fixed position and a second position. The filter is fixed in a position in the part of the container means containing the erythrocytes during the first part of the centrifugation where the leukocytes and thrombocytes has been separated, while the fibrin in the serum has not been compacted. Provided that the density of the filter is less than that of serum, a release of the filter will cause the filter to be transferred to the top of the tube and thereby collecting the leucocyte and thrombocyte layer and compacting the fibrin layer.

In another aspect of the method, the compacting means, such as a filter, is fixed in the first fixed position by a deformation in the container means wall. The filter is fixed by deforming the tube wall. The filter is released by removing the deformation of the wall.

In another aspect of the method, plasma comprising fibrin and a buffy coat comprising leukocytes and thrombocytes are used instead of whole blood. Hereby, a method is provided where whole blood excluding erythrocytes can be used, thus the blood product is directly derivable from whole blood, fractions of whole blood or combinations of fractions of whole blood.

In another aspect, the method is performed within 15 minutes. The method is performed within at least 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 21 minutes, 22 minutes, 23 minutes, 24 minutes, 25 minutes, 26 minutes, 27 minutes, 28 minutes, 29 minutes 30 minutes; 40 minutes, 50 minutes, 60 minutes, 70 minutes, 80 minutes 90 minutes, 100 minutes or even within 120 minutes.

After preparation the blood product can be modified for extended storage before use, such a drying, freezing or freeze-drying, with or without addition of preservation components. This allows generation of blood products for use according to the disclosure well before the surgery so that an operation and can be performed without delay for preparing blood products for use according to the disclosure. Further, a hospital can prepare an adequate supply of blood products from blood sampled provided from volunteers so that blood products are also available even if it was not anticipated before the surgery procedure started.

### BRIEF DESCRIPTION OF THE DRAWINGS

FIG. 1 shows a scientist displaying the single layer patch prepared in Example 1.

FIG. 2 shows a rat where an incision has been made in the abdomen exposing the uterine horns. The contour of the uterine horns is highlighted by lines.

FIG. 3 shows a picture of a surgery on a pig, showing a Patch placed on bleeding lesion created on the liver and the bleeding was stopped after applying the patch.

FIG. 4 shows the progress of healing of a nasal surgical wound using the blood product according to the disclosure.

### DETAILED DESCRIPTION OF THE DISCLOSURE

### Example 1—Preparation of Single Layer Patch

A frozen plasma bag (450 ml) containing human plasma was thawed and calcium chloride was added to initiate coagulation.

After coagulation was completed, determined by visual inspection, the outlet tube was opened in one end and the whole plasma bag was compressed, by placing a flat surface with a heavy weight on the bag.

After compression was complete and the bag was flat, the bag was opened, and the single layer patch removed from the bag.

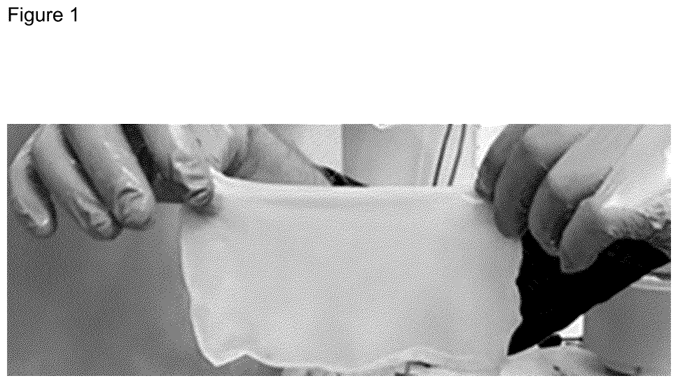

The generated single layer patch is illustrated on FIG. 1.

### Example 2

For this example, blood products in form of circular patches were prepared using the method described in example 3 of U.S. Pat. No. 8,980,301 B, which is incorporated by reference.

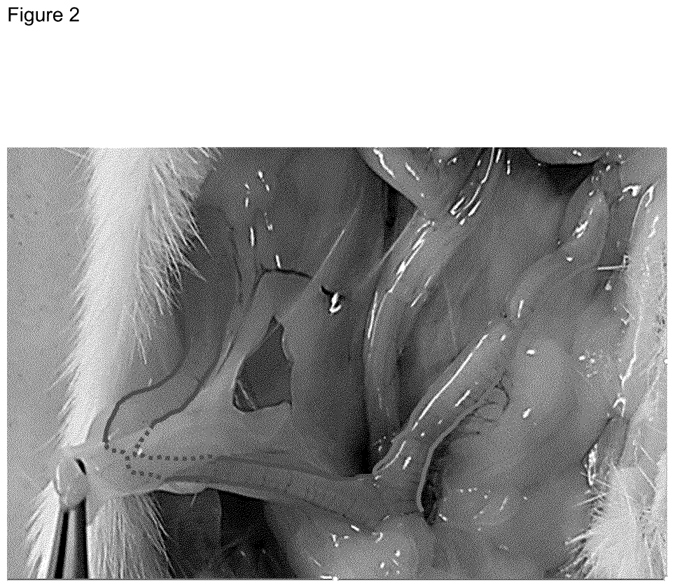

A laboratory rat was used for this example. The rat was anesthetized, and an incision made in the abdomen, so the uterine horns were exposed, see FIG. 2.

The two uterine horns were damaged by cauterizing the uterine horns. On the rat lefts side the lesion was covered with a patch, whereas no covering was applied on the right side.

After one week, the site was evaluated, and adhesion was clear on the right uterine horn that served as control and no adhesion on the left uterine horn treated with the patch.

### Example 3

For this example, blood products in form of circular patches were prepared using the method described in example 5 in U.S. Pat. No. 8,980,301 B, which is incorporated by reference, except that Copolyester(PET) containers were used instead of polyamide containers.

A laboratory pig was used for this example. The pig was anesthetized, and an incision made in the abdomen, so the liver was exposed.

Cuts were made in the liver leading to bleeding from the wounds.

Patches were placed covering the bleeding lesions on the liver which led to a fast cessation of the bleeding, whereafter the surgical procedure could swiftly be resumed.

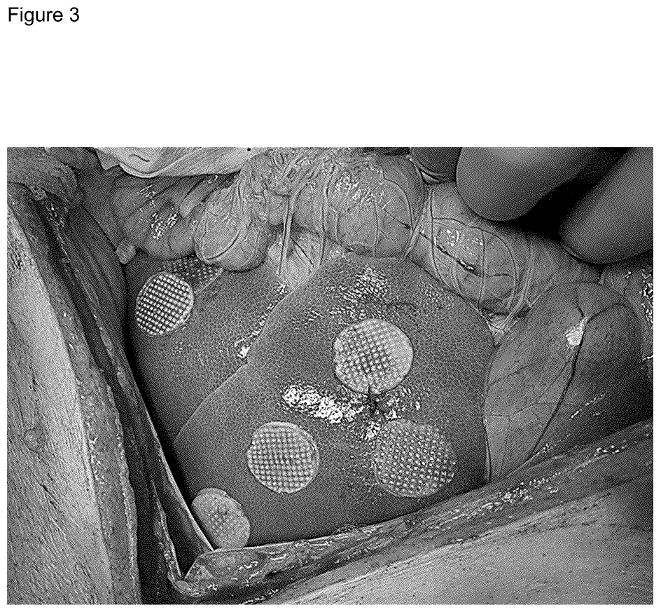

FIG. 3 shows the surgical incision with the patches covering the lesions.

In a control experiment where no patches were placed over the bleeding lesions, bleeding continued and thereby providing a challenge for the continued surgical procedure.

### Example 4—Nasal Skin Cancer Surgical Wound

A patient diagnosed with skin cancer on the nose wing participated in this example.

A blood sample was collected from the patient and a three layer blood product comprising a first layer comprising a majority of leukocytes, and a second layer comprising a majority of thrombocytes and a third layer comprising majority of fibrin was made by centrifugation according to the methods disclosed in WO 2010/020254A1. The blood product was collected as a patch and used for this example.

After the skin cancer was removed by surgery the wound was covered by the patch, securing that the patch was covering the whole wound bed and in contact with the intact skin surrounding the wound, and fastened by suture.

The healing of the wound was followed 3 months after the surgery, and it was seen that the wound healed well and after 3 months no scarring was observed.

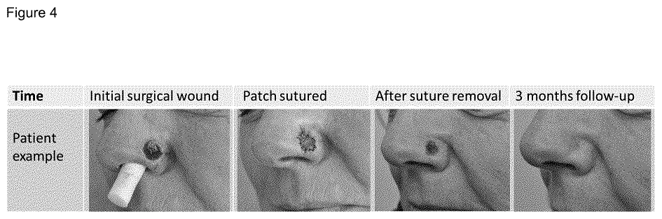

FIG. 4 shows pictures of the wound immediately after the surgery, after the patch has been sutures, after suture removal and after 3 months.

1. A blood product for use in a method for preventing or reducing adhesion during surgery between surfaces where adhesion is to be prevented, comprising the steps of
a. identifying surfaces that during surgery will be located close to each other, which surfaces are not intended to adhere to each other; and
b. applying a blood product comprising a high concentration of fibrin at one or all of the identified surfaces.

2. A blood product for use in a method for surgical wound treatment comprising the steps of
c. Cleaning the wound from foreign material;
d. Removing fluids such as blood;
e. Applying a blood product to the wound, so the blood product covers the complete wound bed ensuring contact between the blood product and the intact skin boundary surrounding the wound; and
f. Covering the wound with a primary sheet;
wherein the blood product is selected among:
i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the wound; and
ii. single layered blood products consisting of components from plasma, components added to initiate a coagulation process and components created during the coagulation process.

3. A blood product for use in a method for stopping or reducing bleeding in an open surgical wound comprising the steps of
g. identifying the bleeding source;
h. removing excess fluids such as blood from the area around the bleeding source;
i. applying a blood product to the bleeding source; and
j. maintaining the blood product in close contact with the bleeding source until bleeding is ceased or reduced;
wherein the blood product is selected among:
i. multilayered blood products comprising components from whole blood, especially thrombocytes and leukocytes, the blood product comprising a first layer, and a second layer, the first layer being adjacent to the second layer, the first layer defining a first outer surface of the blood product, the first layer comprising a majority of leukocytes and the second layer comprising a majority of thrombocytes; and wherein the blood product is oriented so the first layer is in direct contact with the bleeding source; and
ii. single layered blood products consisting of components from plasma, components added to initiate a coagulation process and components created during the coagulation process.

4. The blood product according to any of claims 1-3, wherein the blood product comprise fibrin that have resistance against fibrinolysis.

5. The blood product according to claim 4, wherein the fibrin that have resistance against fibrinolysis comprises fibrin that has been polymerized in 3 dimensions and compressed to a thinner layer sheet.

6. The blood product according to any of the claims 1 to 5, wherein the blood product is reinforced by another structure such as a net (Vicryl net).

7. The blood product according to any of claims 1-6, wherein blood product comprises components from whole blood, especially fibrin, thrombocytes and leukocytes, the blood product comprising a first layer, a second layer and a third layer, the second layer being adjacent to the first layer and the third layer, the first layer defining a first outer surface of the blood product and the third layer defining a second outer surface of the blood product, the first layer comprising a majority of leukocytes, the second layer comprising a majority of thrombocytes and the third layer comprising a majority of fibrin.

8. The blood product according to any of claims 1-7, wherein the blood product consists of components from plasma, components added to initiate a coagulation process and components created during the coagulation process

9. The blood product according to any of claims 1-8, wherein the fibrin, thrombocytes and/or the leukocytes are autologous.

10. The blood product according to any of claims 1-9, wherein the blood product has been modified to extended storage.

11. The blood product according to claim 10, wherein one or more preserving components have been added to the blood product before freezing, freeze drying or drying.

12. A method for preventing or reducing adhesion during surgery between surfaces where adhesion is to be prevented, comprising the steps of
a. identifying surfaces that during surgery will be located close to each other, which surfaces are not intended to adhere to each other; and
b. applying a blood product comprising a high concentration of fibrin at one or all of the identified surfaces.

13. The method of claim 12, wherein the blood product comprise fibrin that have resistance against fibrinolysis.

14. The method of claim 13, wherein the fibrin that have resistance against fibrinolysis comprises fibrin that has been polymerized in 3 dimensions and compressed to a thinner layer sheet.

15. The method according to any of the claims 12-14, wherein the blood product is reinforced by another structure such as a net (Vicryl net).

16. The method according to claim 12, wherein blood product comprising components from whole blood, especially fibrin, thrombocytes and leukocytes, the blood product comprising a first layer, a second layer and a third layer, the second layer being adjacent to the first layer and the third layer, the first layer defining a first outer surface of the blood product and the third layer defining a second outer surface of the blood product, the first layer comprising a majority of leukocytes, the second layer comprising a majority of thrombocytes and the third layer comprising a majority of fibrin.

17. The method according to claim 12, wherein the blood product consists of components from plasma, components added to initiate a coagulation process and components created during the coagulation process.

18. The method of any of claims 12-17, wherein the fibrin, thrombocytes and/or the leukocytes are autologous.

19. The method according to any of claims 12-18, wherein the blood product has been modified to extended storage.

20. The method according to claim 19, wherein the blood product has been extended by freezing, freeze during or drying.

21. The method according to claim 20, wherein one or more preserving components have been added to the blood product before freezing, freeze drying or drying.
